(19) United States
(12) Patent Application Publication
(10) Pub. No.: US 2020/0038190 A1
(43) Pub. Date: Feb. 6, 2020

(54) ACETABULAR PROSTHESIS INCLUDING MODULAR COMPONENTS AND METHOD

(71) Applicant: Depuy Synthes Products, Inc., Raynham, MA (US)

(72) Inventors: Larry G. McCleary, Warsaw, IN (US); James Brooks, Burley in Wharfedale (GB)

(21) Appl. No.: 16/051,329

(22) Filed: Jul. 31, 2018

(51) Int. Cl. A61F 2/34; A61F 2/36; A61F 2/46; A61F 2/30

(52) U.S. Cl. CPC A61F 2/34; A61F 2/3609; A61B 17/86; A61F 2/30734; A61F 2/4609

(57) ABSTRACT

An acetabular orthopaedic prosthesis and method are disclosed. The acetabular orthopaedic prosthesis includes a shell configured to engage a surgically-prepared surface of a patient's acetabulum. The shell includes one or more mounting holes that extend through the inner surface and the outer surface. A plurality of plugs are configured to be coupled to the shell, and each plug is sized to be positioned in at least one of the mounting holes of the shell. A bone screw is configured to be inserted through one of the plurality of plugs into the surgically-prepared surface to secure the shell to the patient's acetabulum.

### TECHNICAL FIELD

The present disclosure relates generally to orthopaedic prosthetic components and, more particularly, to acetabular prosthetic components.

### BACKGROUND

Joint arthroplasty is a well-known surgical procedure by which a diseased and/or damaged natural joint is replaced by a prosthetic joint. For example, in a hip arthroplasty surgical procedure, a patient's natural hip ball and socket joint is partially or totally replaced by a prosthetic hip joint. A typical prosthetic hip joint includes an acetabular prosthetic component and a femoral head prosthetic component. An acetabular prosthetic component generally includes an outer shell configured to engage the acetabulum of the patient and an inner bearing or insert coupled to the shell and configured to engage the femoral head. The femoral head prosthetic component and inner insert of the acetabular component form a ball and socket joint that approximates the natural hip joint.

In some acetabular prosthetic components, the outer shell is secured to the patient's acetabulum via one or more bone screws. The outer shell may include one or more circular or elongated openings through which the bone screw may be inserted to engage the patient's acetabulum.

### SUMMARY

According to one aspect, an acetabular orthopaedic prosthesis is disclosed. The acetabular orthopaedic prosthesis comprises an insert component including a cavity sized to receive a head of a femoral prosthetic component and a shell including a convex outer surface shaped to engage a surgically-prepared surface of a patient's acetabulum. The shell also includes a concave inner surface that defines an aperture sized to receive the insert and a plurality of mounting holes extending through the inner surface and the outer surface. The acetabular orthopaedic prosthesis further comprises a plurality of plugs configured to be coupled to the shell and a bone screw configured to be inserted through one of the plurality of plugs into the surgically-prepared surface to secure the shell to the patient's acetabulum. Each plug is sized to be positioned in at least one of the mounting holes of the shell.

In some embodiments, the plurality of mounting holes may include a number of elongated slots, and the plurality of plugs may include an elongated plug sized to be positioned in a first elongated slot of the number of elongated slots. The elongated plug may include a guide hole sized to receive the bone screw. The guide hole may have a diameter that is smaller than a length of the first elongated slot.

In some embodiments, the elongated plug may be a first elongated plug having a first guide hole configuration that includes the guide hole sized to receive the bone screw, and the plurality of plugs may include a second elongated plug sized to be positioned in a second elongated slot of the number of elongated slots. The second elongated plug may have a second guide hole configuration that is different from the first guide hole configuration.

Additionally, in some embodiments, the second elongated plug may include a plurality of guide holes in the second guide hole configuration. Each guide hole of the second elongated plug may be sized to receive a bone screw. In some embodiments, the second elongated plug may be devoid of guide holes in the second guide hole configuration.

In some embodiments, the elongated plug may include a concave curved surface shaped to match a curvature of the concave inner surface of the shell.

In some embodiments, the shell may be a metallic shell and each plug of the plurality of plugs may be a polymeric plug.

Additionally, in some embodiments, the plurality of plugs may include an insert plate sized to cover a number of mounting holes of the plurality of mounting holes. The insert plate may include a concave curved surface shaped to match a curvature of the concave inner surface of the shell. In some embodiments, the insert plate may include a number of openings sized to receive the bone screw and a peg removably positioned in and filling each opening. Each opening of the insert plate may be aligned with one of the mounting holes of the plurality of mounting holes.

In some embodiments, the plurality of plugs may include a first plug having a base sized to be positioned in one of the mounting holes of the shell and a shaft coupled to the base. The shaft may be configured to extend outwardly from the outer surface of the shell to a tip configured to engage the patient's acetabulum.

Additionally, in some embodiments, the plurality of plugs may include a first plug including a frangible seam configured to open to permit the bone screw to be inserted through the first plug into the surgically-prepared surface.

According to another aspect of the disclosure, a method of implanting an acetabular orthopaedic prosthesis is disclosed. The method comprises inserting a shell of the acetabular orthopaedic prosthesis into an acetabulum of a patient's pelvic bone. The shell includes a mounting hole extending through an outer surface and an inner surface of the shell. The method also comprises inserting a bone screw through a plug positioned in the mounting hole into a surgically-prepared surface of the patient's acetabulum to secure the shell to the patient's acetabulum.

In some embodiments, the method may further comprise identifying a location on the surgically-prepared surface of the patient's acetabulum to insert the bone screw, selecting a plug having a guide hole corresponding to the identified location, and attaching the plug to the shell. The step of inserting the bone screw through the plug may include inserting the bone screw through the guide hole in the plug into the identified location on the surgically-prepared surface.

In some embodiments, the mounting hole may be an elongated slot, and the guide hole may have a diameter that is smaller than a length of the first elongated slot.

Additionally, in some embodiments, the shell may include a plurality of mounting holes, and the plug may include a plate that covers a number of mounting holes of the plurality of mounting holes. In some embodiments, the step of inserting the bone screw through the plug may include selecting one mounting hole of the number of mounting holes and inserting the bone screw through the selected mounting hole into the surgically-prepared surface of the patient's acetabulum.

In some embodiments, the method may further comprise detaching a portion of the plug to expose a guide hole of the plug. The step of inserting the bone screw through the plug may include inserting the bone screw through the guide hole in the plug into the identified location on the surgically-prepared surface.

Additionally, in some embodiments, the step of inserting the shell of the acetabular orthopaedic prosthesis into the acetabulum of the patient's pelvic bone may include engaging a tip of the plug extending away from the outer surface of the shell with the surgically-prepared surface of the patient's acetabulum.

In some embodiments, the step of inserting the bone screw through the plug may include engaging a tip of the bone screw with a frangible seam of the plug to open the seam and advancing the tip of the bone screw through the open seam.

According to another aspect, an acetabular orthopaedic prosthesis is disclosed. The acetabular orthopaedic prosthesis comprises an insert component including a cavity sized to receive a head of a femoral prosthetic component and a shell including a convex outer surface shaped to engage a surgically-prepared surface of a patient's acetabulum, a concave inner surface that defines an aperture sized to receive the insert, and a mounting hole extending through the inner surface and the outer surface. The acetabular orthopaedic prosthesis comprises a plug configured to be coupled to the shell. The plug is sized to be positioned in the mounting holes of the shell and includes a concave surface having a curvature that matches a curvature of the concave inner surface of the shell. The prosthesis also comprises a bone screw configured to be inserted through the plugs into the surgically-prepared surface to secure the shell to the patient's acetabulum.

In some embodiments, the plug may include a plurality of guide holes, each guide hole being sized to receive the bone screw.

According to another aspect, a method of implanting an acetabular orthopaedic prosthesis comprises inserting a shell of the acetabular orthopaedic prosthesis into an acetabulum of a patient's pelvic bone. The shell includes a number of mounting holes extending through an outer surface and an inner surface of the shell. The method also comprises identifying a number of locations on a surgically-prepared surface of the patient's acetabulum to insert a number of bone screws, selecting a first guide hole of a first plug corresponding to a first identified location of the number of locations, inserting a first bone screw through the first guide hole of the first plug positioned in a first mounting hole of the number of mounting holes into the surgically-prepared surface of the patient's acetabulum, selecting a second guide hole of a second plug corresponding to a second identified location of the number of locations, and inserting a second bone screw through the second guide hole of the second plug positioned in a first mounting hole of the number of mounting holes into the surgically-prepared surface of the patient's acetabulum.

### BRIEF DESCRIPTION OF THE DRAWINGS

The detailed description particularly refers to the following figures, in which:

FIG. 1 is an exploded perspective view of a hip orthopaedic prosthesis assembly including an acetabular prosthetic component;

FIG. 2 is an exploded perspective view of a shell assembly of the acetabular prosthetic component of FIG. 1;

FIG. 3 is a cross-sectional elevation view of a shell component taken along the line 3-3 in FIG. 2;

FIG. 4 is a bottom perspective view of a plug of the shell assembly of FIG. 2;

FIG. 5 is a bottom perspective view of another plug of the shell assembly of FIG. 2;

FIG. 6 is another perspective view of the plug of FIG. 5;

FIGS. 7-8 are perspectives view of another plug of the shell assembly of FIG. 2;

FIG. 9 is a perspective view of the shell component of FIGS. 2-3 implanted with the plugs of FIGS. 4-6 into a surgically prepared acetabulum of a patient;

FIG. 10 is a perspective view of the shell component of FIGS. 2-3 implanted with the plugs of FIGS. 5-8 into a surgically prepared acetabulum of a patient;

FIG. 11 is a perspective view of the shell component of FIGS. 2-3 and another embodiment of a plug;

FIG. 12 is an exploded perspective view of a shell assembly of a hip orthopaedic prosthesis assembly;

FIG. 13 is a perspective view of a shell assembly of a hip orthopaedic prosthesis assembly;

FIG. 14 is a perspective view of a shell assembly of a hip orthopaedic prosthesis assembly; and

FIGS. 15-16 are perspective views of a shell assembly of a hip orthopaedic prosthesis assembly.

### DETAILED DESCRIPTION OF THE DRAWINGS

While the concepts of the present disclosure are susceptible to various modifications and alternative forms, specific exemplary embodiments thereof have been shown by way of example in the drawings and will herein be described in detail. It should be understood, however, that there is no intent to limit the concepts of the present disclosure to the particular forms disclosed, but on the contrary, the intention is to cover all modifications, equivalents, and alternatives falling within the spirit and scope of the invention as defined by the appended claims.

Terms representing anatomical references, such as anterior, posterior, medial, lateral, superior, inferior, etcetera, may be used throughout the specification in reference to the orthopaedic implants or prostheses and surgical instruments described herein as well as in reference to the patient's natural anatomy. Such terms have well-understood meanings in both the study of anatomy and the field of orthopaedics. Use of such anatomical reference terms in the written description and claims is intended to be consistent with their well-understood meanings unless noted otherwise.

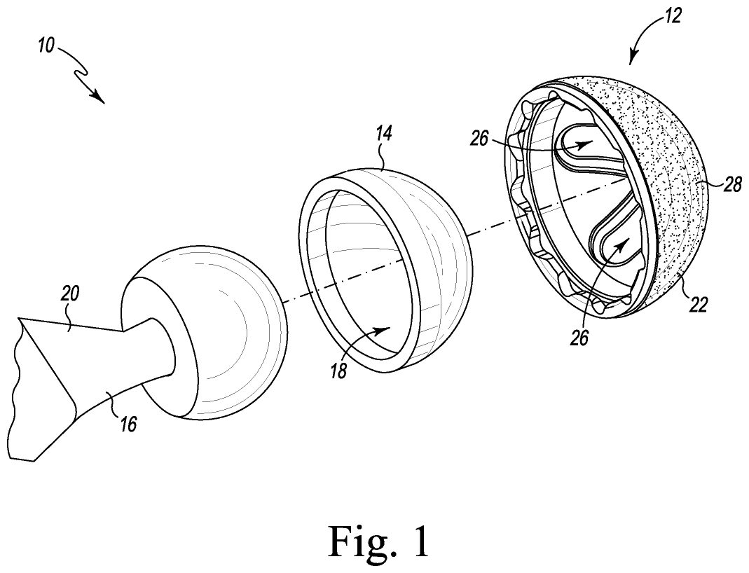

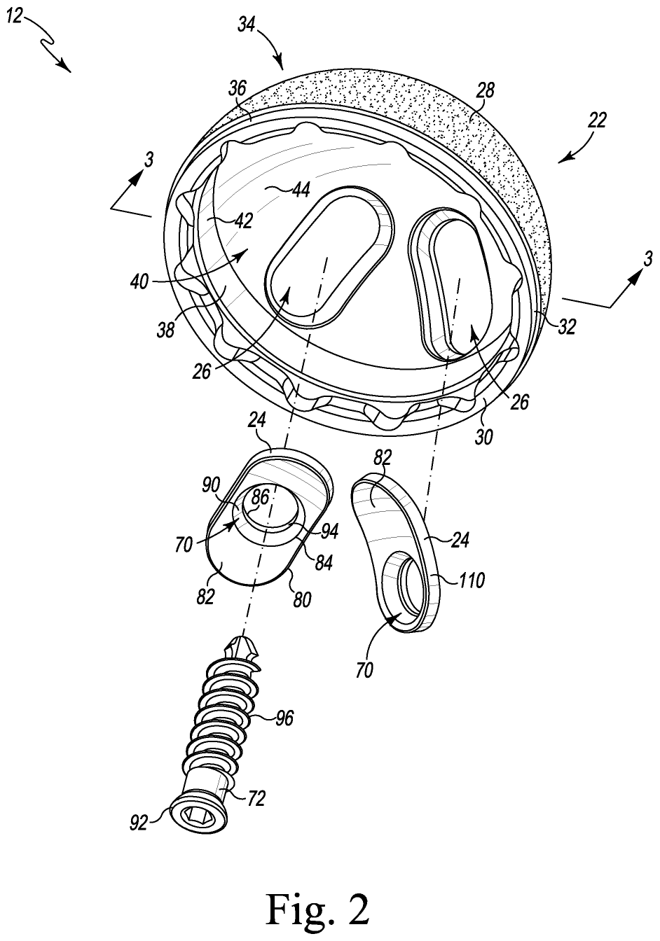

Referring now to FIG. 1, a hip orthopaedic prosthesis assembly 10 includes a plurality of components, including an acetabular shell assembly 12 that is sized to be implanted in a surgically-prepared acetabulum of a patient's pelvis. The assembly 10 also includes an acetabular insert component 14 configured to be coupled to the shell assembly 12 and a femoral head component 16 that is configured to be received in a cavity 18 defined in the insert 14. The prosthesis assembly 10 also includes a femoral stem component 20 is configured to be secured to the femoral head component 16. In the illustrative embodiment, the acetabular shell assembly 12 includes a shell component 22 that is configured to receive the insert 14. As described in greater detail below, the shell assembly 12 also includes a number of other modular components, including plugs 24 (see FIG. 2), which are sized and shaped to be positioned in the mounting holes 26 defined in the shell component 22 to seal or partially fill each hole 26.

The hip orthopaedic prosthesis assembly 10 is formed from a variety of different materials. The insert 14 is illustratively formed from a polymeric material such as, for example, polyethylene. The shell component 22, femoral head component 16, and femoral stem component 20 are separately formed from implant-grade metallic materials such as, for example, cobalt chromium. The shell 22 also includes a Porocoat® outer coating 28 that permits bone to affix biologically to the shell 22 after implantation. It should be appreciated that in other embodiments the Porocoat® outer coating may be omitted. In the illustrative embodiment, each plug 24 is also formed from an implant-grade metallic material, but it should be appreciated that in other embodiments the plugs 24 may be formed from a polymeric material such as, for example, polyethylene.

Referring now to FIG. 2, the shell component 22 has a distal rim 30 and an outer wall 32 that extends from the distal rim 30. The outer wall 32 includes a convex curved outer surface 34 and an annular outer surface 36 that extends from the distal rim 30 to the curved outer surface 34. In the illustrative embodiment, the convex curved outer surface 34 is semi-spherical, and the Porocoat® outer coating 28 covers the outer surface 34.

An inner wall 38 extends inwardly from the distal rim 30 to define a cavity 40 sized to receive the insert component 14. The inner wall 38 includes an annular inner surface 42 that is positioned opposite the annular outer surface 36 and a concave curved inner surface 44 that is connected to the annular inner surface 42. In the illustrative embodiment, the shell component 22 has a pair of mounting holes 26 that extend through the outer surface 34 and the inner surface 44.

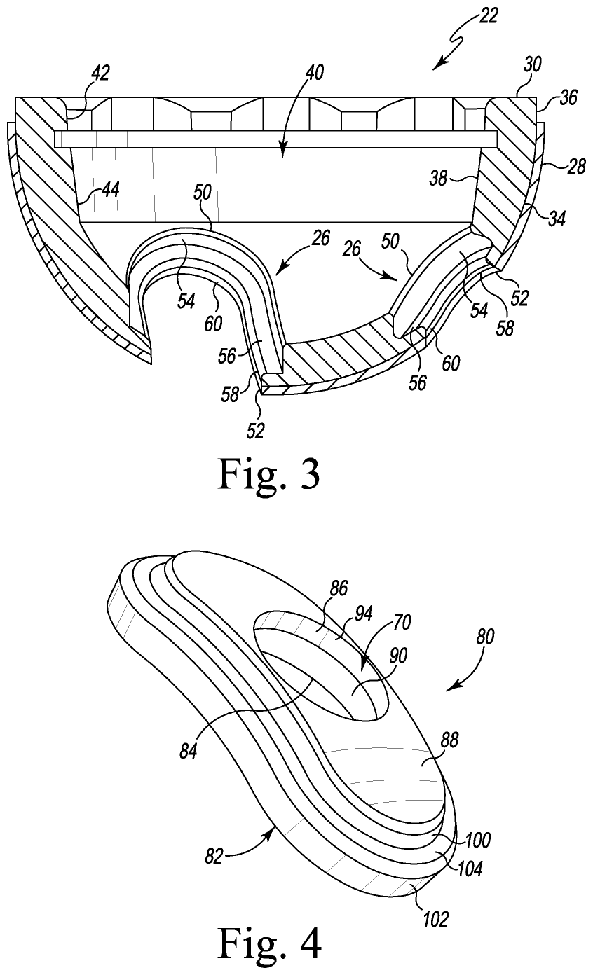

Referring now to FIG. 3, each mounting hole 26 has an inner opening 50 defined in the inner surface 44 and an outer opening 52 defined in the outer surface 34 and the outer coating 28. An inner wall 54 extends outwardly from the inner opening 50 to a ledge surface 56 that is positioned between the surfaces 34, 44. The ledge surface 56 defines another opening 58, and the shell component 22 has an inner wall 60 that extends outwardly from each opening 58 to each outer opening 52. The inner walls 54, 60 and the ledge surface 56 cooperate to define each mounting hole 26. In the illustrative embodiment, each mounting hole 26 is an elongated slot having closed, curved ends. It should be appreciated that in other embodiments one or both of the mounting holes may take other geometric forms such as, for example, a circular hole. It should also be appreciated that in other embodiments the shell component 22 may include only a single mounting hole or more than two mounting holes.

Returning to FIG. 2, the shell assembly 12 includes a pair of plugs 24, which are sized and shaped to be positioned in the mounting holes 26. In the illustrative embodiment, each plug 24 includes a fastener guide hole 70 that is sized and shaped to guide the insertion of a fastener, such as, for example, bone screw 72, into the patient's bone to secure the shell component 22 to the patient's bone. As described in greater detail below, the location of the guide hole 70 in each plug 24 is different such that each plug has a distinct guide hole configuration. It should be appreciated that in other embodiments other a plug may have another guide hole configuration. For example, the guide hole may be omitted from one of the plugs 24 such that the plug is devoid of guide holes. Additionally, although the plugs 24 are sized to fill and seal mounting holes 26, in other embodiments one or both of the plugs 24 may be sized to only partially fill their respective mounting holes 26.

The pair of plugs 24 includes an elongated, oblong plug 80 that has a concave curved inner surface 82 and a fastener guide hole 70 that is positioned in the middle of the surface 82. In the illustrative embodiment, the curvature of the concave curved inner surface 82 matches the curvature of the concave curved inner surface 44 of the shell component 22 and is configured to be positioned flush with the inner surface 44 when the plug 80 is positioned in one of the mounting holes 26. It should be appreciated that in other embodiments the plugs 24 may be configured to be recessed below the inner surface 44 of the shell component 22.

The plug 80 includes an inner opening 84 that is defined in the inner surface 82, and an inner wall 86 extends outwardly from the inner opening 84 to an opening defined in the outer surface 88 (see FIG. 4) of the plug 80. In the illustrative embodiment, the inner wall 86 includes a conical surface 90 that extends from the inner surface 82 to define a countersink of the fastener guide hole 70 sized to receive the head 92 of the bone screw 72. The inner wall 86 also has an annular surface 94 that defines a bore sized to receive the threaded shaft 96 of the bone screw 72.

Referring now to FIG. 4, the plug 80 includes an outer base 100 that is sized and shaped to extend through the opening 58 defined by the ledge surface 56 in each mounting hole 26 of the shell component 22. The outer base 100 includes the outer surface 88, which is illustrative a convex curved surface having a curvature that matches the curvature of the convex curved outer surface 34 of the shell component 22. The plug 80 also includes an inner body 102, which extends from the base 100 and is sized to be positioned in the inner opening 50 of each mounting hole 26 of the shell component 22. The inner body 102 includes a flange surface 104 that extends around the outer base 100 and is configured to engage a ledge surface 56 of the shell component 22 when the plug 80 is positioned in the corresponding mounting hole 26.

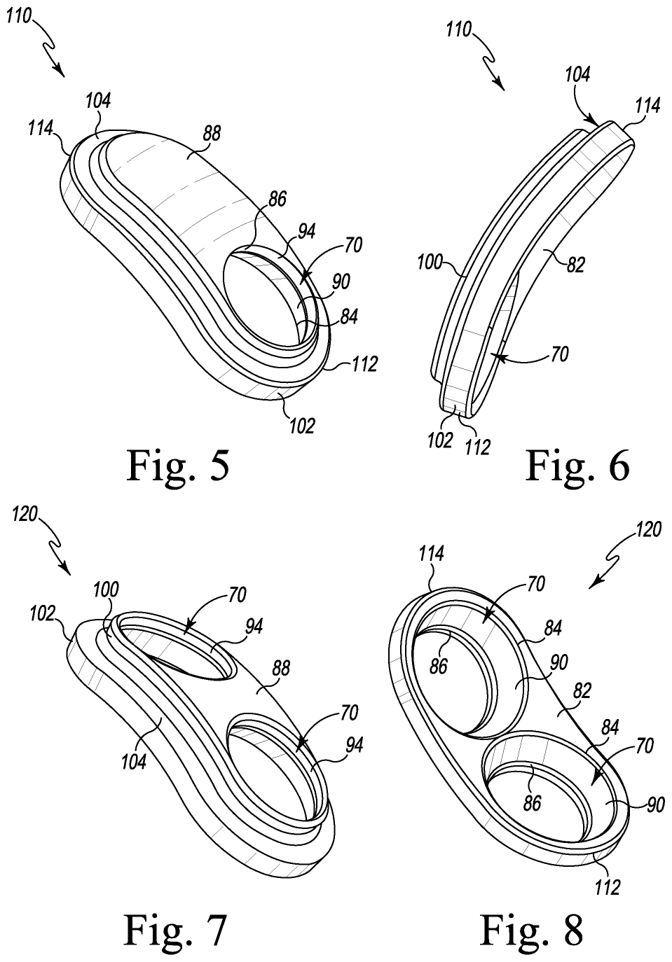

Referring now to FIGS. 5-6, the other plug of the pair of plugs 24 is an elongated, oblong plug 110 that, like the plug 80, has a concave curved inner surface 82 that is configured to be positioned flush with the inner surface 44 when the plug 110 is positioned in one of the mounting holes 26. In the illustrative embodiment, the surfaces 82 of the plugs 80, 110 cooperate with the inner surface 44 of the shell component 22 to define a single concave curved inner surface of the shell assembly 12 when the plugs 80, 110 are seated in their respective mounting holes 26. It should also be appreciated that either of the plugs 80, 110 may be positioned in either of the mounting holes 26.

In the illustrative embodiment, the oblong plug 110 extends from an end 112 to another end 114, and the fastener guide hole 70 of the plug 110 is positioned at the end 112 of the plug 110. The plug 110 is configured to be reversible in the mounting holes 26 so that the fastener guide hole 70 may be positioned at either end of the mounting hole 26 in which the plug 110 is positioned. To define the fastener guide hole 70, the plug 110, like the plug 80, includes an inner opening 84 that is defined in the inner surface 82, and an inner wall 86 extends outwardly from the inner opening 84 to an opening defined in the outer surface 88 of the plug 110. In the illustrative embodiment, the inner wall 86 includes a conical surface 90 that extends from the inner surface 82 to define a countersink of the fastener guide hole 70 sized to receive the head 92 of the bone screw 72. The inner wall 86 also has an annular surface 94 that defines a bore sized to receive the threaded shaft 96 of the bone screw 72.

As shown in FIG. 6, the plug 110 includes an outer base 100 that is sized and shaped to extend through the opening 58 defined by the ledge surface 56 in each mounting hole 26 of the shell component 22. The outer base 100 includes the outer surface 88, which is illustrative a convex curved surface having a curvature that matches the curvature of the convex curved outer surface 34 of the shell component 22. The plug 110 also includes an inner body 102, which extends from the base 100 and is sized to be positioned in the inner opening 50 of each mounting hole 26 of the shell component 22. The inner body 102 includes a flange surface 104 that extends around the outer base 100 and is configured to engage a ledge surface 56 of the shell component 22 when the plug 110 is positioned in the corresponding mounting hole 26.

It should be appreciated that the shell assembly 12 may be included in a prosthesis system or kit that includes additional plugs 24 having various guide hole configurations. For example, the system may include at least one other plug 110 so that the assembly 12 is assembled with a fastener guide hole 70 positioned at an end of each mounting hole 26. The system may include at least one other plug 80 so that the assembly 12 is assembled with a fastener guide hole 70 positioned in the middle of each mounting hole 26. In still other embodiments, the prosthesis system may include one or more elongated, oblong plugs 120, as shown in FIGS. 7-8.

The oblong plug 120 has a fastener guide hole configuration that is different from the guide hole configurations of plugs 80, 110. Like the plugs 80, 110, the plug 120 has a concave curved inner surface 82 that is configured to be positioned flush with the inner surface 44 when the plug 120 is positioned in one of the mounting holes 26. In the illustrative embodiment, the oblong plug 120 extends from an end 112 to another end 114. The guide hole configuration of the plug 120 includes a fastener guide hole 70 positioned at each of the ends 112, 114. The plug 120, like the plugs 80, 110, is sized to fill and seal its mounting hole 26. It should be appreciated that in other embodiments the plug 120 may be sized to only partially fill the mounting hole 26.

To define each fastener guide hole 70, the plug 120, like the plugs 80, 110, includes an inner opening 84 that is defined in the inner surface 82, and an inner wall 86 extends outwardly from the inner opening 84 to an opening defined in the outer surface 88 of the plug 120. In the illustrative embodiment, the inner wall 86 includes a conical surface 90 that extends from the inner surface 82 to define a countersink of the fastener guide hole 70 sized to receive the head 92 of the bone screw 72. The inner wall 86 also has an annular surface 94 that defines a bore sized to receive the threaded shaft 96 of the bone screw 72.

As shown in FIG. 7, the plug 110 includes an outer base 100 that is sized and shaped to extend through the opening 58 defined by the ledge surface 56 in each mounting hole 26 of the shell component 22. The outer base 100 includes the outer surface 88, which is illustrative a convex curved surface having a curvature that matches the curvature of the convex curved outer surface 34 of the shell component 22. The plug 120 also includes an inner body 102, which extends from the base 100 and is sized to be positioned in the inner opening 50 of each mounting hole 26 of the shell component 22. The inner body 102 includes a flange surface 104 that extends around the outer base 100 and is configured to engage a ledge surface 56 of the shell component 22 when the plug 120 is positioned in the corresponding mounting hole 26.

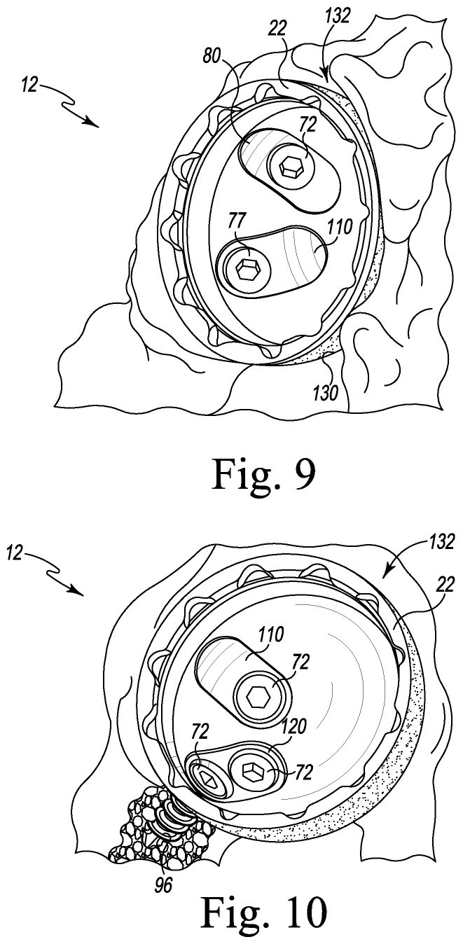

As shown in FIGS. 9-10, a surgeon inserts a shell component 22 into a surgically-prepared surface 130 of the patient's acetabulum 132 with the plugs 24 selected by the surgeon. It should be appreciated that the plugs 24 may be attached to the shell component 22 prior to inserting the shell component 22 or after the shell component 22 is seated in the acetabulum. The surgeon may rotate the shell component 22 within the acetabulum to identify locations on the surgically-prepared surface at which to insert a bone screw 72. By rotating of the shell component 22 within the acetabulum 132 and/or changing the plugs 24 inserted into the shell component 22, the surgeon may adjust the direction and location of the fastener guide holes 70 to ensure the proper placement of the bone screws 72 relative to the patient's bone. It should be appreciated that each fastener guide hole 70 of the plugs 80, 110, 120, the fastener guide holes 70 may be tilted or angled to guide the bone screw 72 in a particular direction into the patient's bone. The prosthesis system or kit may therefore include multiple plugs 80, 110, or 120 to change having fastener guide holes 70 of various angles or tilts to accommodate variations in the bony anatomy of the potential patient population. As described and shown in FIGS. 9-10, the elongated plugs 80, 110, 120 seal the mounting holes 26 when the shell assembly 12 is implanted.

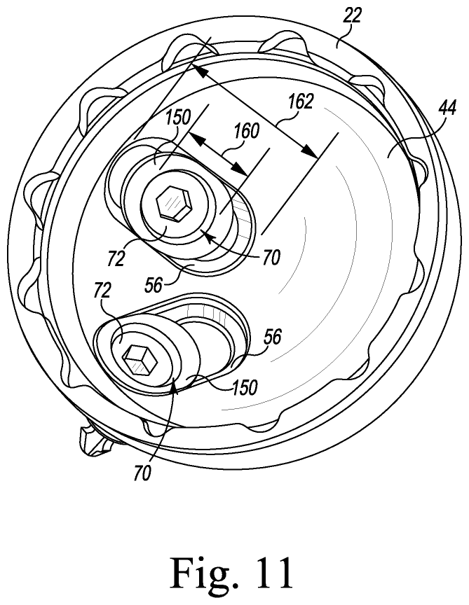

Referring now to FIG. 11, another embodiment of a plug (hereinafter plug 150) is shown. Each plug 150 is sized and shaped to be positioned in each mounting hole 26 defined in the shell component 22. In the illustrative embodiment, the basic configuration of each plug 150 matches the configurations of the plugs 80, 110, 120 (including, for example, an inner curved surface 82, a fastener guide hole 70, and base (not shown)), but the plugs 150 are smaller than the plugs 80, 110, 120 and only partially fill each mounting hole 26. Because each plug 150 is smaller than each mounting hole 26, each plug 150 may be moved along the mounting hole 26 to change the placement of the bone screw 72 to any location along the mounting hole 26.

As shown in FIGS. 1-11, each fastener guide hole 70 of the plugs 80, 110, 120, 150 has a diameter 160 that is defined by its annular surface 94. The diameter 160 is sized to permit the passage of the threaded shaft 96 of a bone screw 72 through the fastener guide hole 70 and into the patient's bone. Each mounting hole 26 of the shell component 22 has a length 162 that is defined between its closed ends (see, e.g., FIG. 11). In the illustrative embodiment, the diameter 160 of the fastener guide hole 70 (and hence the diameter of the threaded shaft 96) is less than the length 162 such that the surgeon is permitted to move the screw between multiple positions and orientations to insert the screw into a desirable location in the patient's bone.

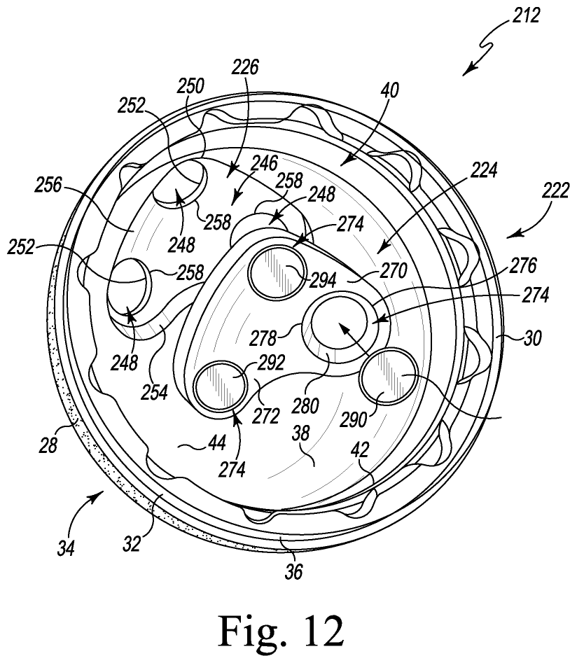

Referring now to FIG. 12, another embodiment of an acetabular shell assembly (hereinafter shell assembly 212 is shown). A number of the features of the shell assembly 212 are the same as features shown and described above in regard to the shell assembly 12. Those features are identified in FIG. 12 using the same reference numbers. The shell assembly 212 includes a shell component 222 that is configured to receive the insert 14. As described in greater detail below, the shell assembly 212 also includes a plug 224, which is sized and shaped to be positioned in a mounting hole 226 defined in the shell component 222, to guide a bone screw into contact with the patient's bone.

The shell component 222 has a distal rim 30 and an outer wall 32 that extends from the distal rim 30. The outer wall 32 includes a convex curved outer surface 34 and an annular outer surface 36 that extends from the distal rim 30 to the curved outer surface 34. In the illustrative embodiment, the convex curved outer surface 34 is semi-spherical, and the Porocoat® outer coating 28 covers the outer surface 34.

An inner wall 38 extends inwardly from the distal rim 30 to define a cavity 40 sized to receive the insert component 14. The inner wall 38 includes an annular inner surface 42 that is positioned opposite the annular outer surface 36 and a concave curved inner surface 44 that is connected to the annular inner surface 42. In the illustrative embodiment, the mounting hole 226 includes a recess 246 that is defined in the inner surface 44 and a number of through holes 248 that extend from the recess 246 to the outer surface 36.

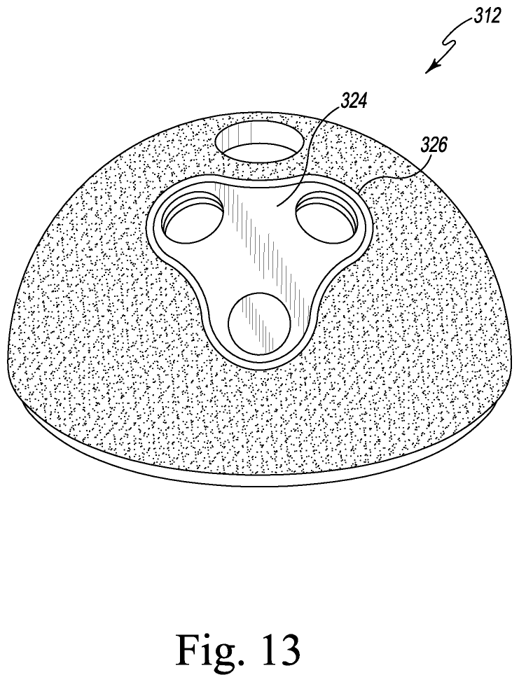

The mounting hole 226 has an inner opening 250 defined in the inner surface 44 and a number of outer openings 252 defined in the outer surface 34 and the outer coating 28. An inner wall 254 extends outwardly from the inner opening 250 to a ledge surface 256 that is positioned between the surfaces 34, 44. The ledge surface 256 defines another set of openings 258, and the shell component 222 has an inner wall 260 that extends outwardly from each opening 258 to each outer opening 252. Each inner wall 254 defines a separate through hole 248 in the shell component 22. In the illustrative embodiment, each through hole 248 is circular in shape. It should be appreciated that in other embodiments one or more of the through holes may take other geometric forms such as, for example, an elongated, oblong slot. It should also be appreciated that in other embodiments such as, for example, the shell component 322 shown in FIG. 13, the shell component 322 may include only a single mounting hole 326 sized to receive a single plug 324.

Returning to FIG. 12, the plug 224 of the shell assembly 212 includes an insert plate 270 sized to be positioned in the recess 246 of the mounting hole 226 to cover the through holes 248. The insert plate 270 includes an concave curved inner surface 272 that matches the curvature of the concave curved inner surface 44 of the shell component 222 and is configured to be positioned flush with the inner surface 44 when the insert plate 270 is positioned in the recess 246. It should be appreciated that in other embodiments the insert plate may be configured to be recessed below the inner surface 44 of the shell component 222. Additionally, although the plug 224 is sized to fill and seal the mounting hole 226, in other embodiments the plug 224 may be sized to only partially fill the mounting hole 226.

The plug 224 includes a number of fastener guide holes 274 that is defined in the curved inner surface 272 of the insert plate 270. The fastener guide hole 274 is sized and shaped to guide the insertion of a fastener, such as, for example, the bone screw 72, into the patient's bone to secure the shell component 222 to the patient's bone. Each fastener guide hole 274 is positioned in the insert plate 270 to be aligned with a corresponding through hole 248 when the insert plate 270 is positioned in the recess 246 of the shell assembly 212.

Each fastener guide hole 274 extends from an inner opening 276 defined in the curved inner surface 272 of the insert plate 270 to an outer opening 278 defined in the outer surface (not shown) of the plate 270. A conical surface 280 extends from the inner surface 272 to define a countersink of each fastener guide hole 274 sized to receive the head 92 of the bone screw 72. In the illustrative embodiment, each through hole 248 is sized to permit the passage of the shaft 96 of a bone screw 72.

The plug 224 further includes a number of pegs 290 that are sized to be positioned in each fastener guide hole 274. Each peg 290 is sized to fill and seal the fastener guide hole 274, as indicated by pegs 292, 294 shown in FIG. 12. Each peg 290 is also removable from the fastener guide hole 274 so that the surgeon may utilize the corresponding fastener guide hole 274 during implantation. It should be appreciated that in other embodiments the insert plate may be configured to permit the surgeon to drill through the insert plate to define a fastener guide hole over a corresponding through hole 248.

In the illustrative embodiment, the plug 224, including the insert plate 270 and the pegs 290, is formed from a polymeric material such as, for example, polyethylene. It should be appreciated that in other embodiments the insert plate 270 and/or the pegs 290 may be formed from an implant-grade metallic material.

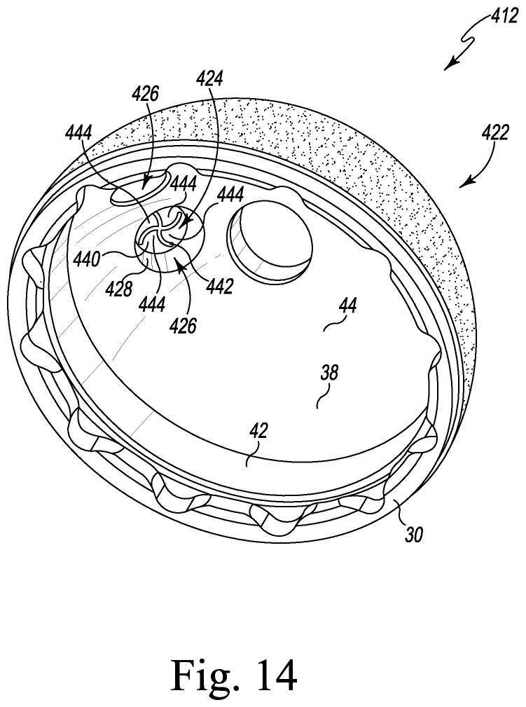

Referring now to FIG. 14, another embodiment of an acetabular shell assembly (hereinafter shell assembly 412 is shown). A number of the features of the shell assembly 412 are the same as features shown and described above in regard to shell assembly 12. Those features are identified in FIG. 14 using the same reference numbers. The shell assembly 412 includes a shell component 422 that is configured to receive the insert 14. As described in greater detail below, the shell assembly 412 also includes a number of plugs 424, which are sized and shaped to be positioned in the mounting holes 426 defined in the shell component 422.

The shell component 422 has a distal rim 30 and an inner wall 38 extends inwardly from the distal rim 30 to define a cavity 40 sized to receive the insert component 14. The inner wall 38 includes an annular inner surface 42 that is positioned opposite the annular outer surface 36 and a concave curved inner surface 44 that is connected to the annular inner surface 42

Each mounting hole 426 is defined by an inner wall 428 that extends through the shell component 422. In the illustrative embodiment, each mounting hole 426 is circular in shape. It should be appreciated that in other embodiments one or more of the mounting holes may take other geometric forms such as, for example, an elongated, oblong slot. It should also be appreciated that in other embodiments the shell component 422 may include additional mounting holes.

Each plug 424 includes a disk-shaped body 440 that is sized to engage the inner wall 428 of the shell component 422 when the plug 424 is positioned in the mounting hole 426. In the illustrative embodiment, each plug 424 is formed from a polymeric material such as, for example, polyethylene. The disk-shaped body 440 includes a central opening 442 that is sized to receive the shaft 96 of a bone screw 72. In the illustrative embodiment, the central opening 442 is sealed by a number of segments 444 of the plug 424, which are connected by frangible seams that are configured to rip or tear open to permit passage of the shaft 96.

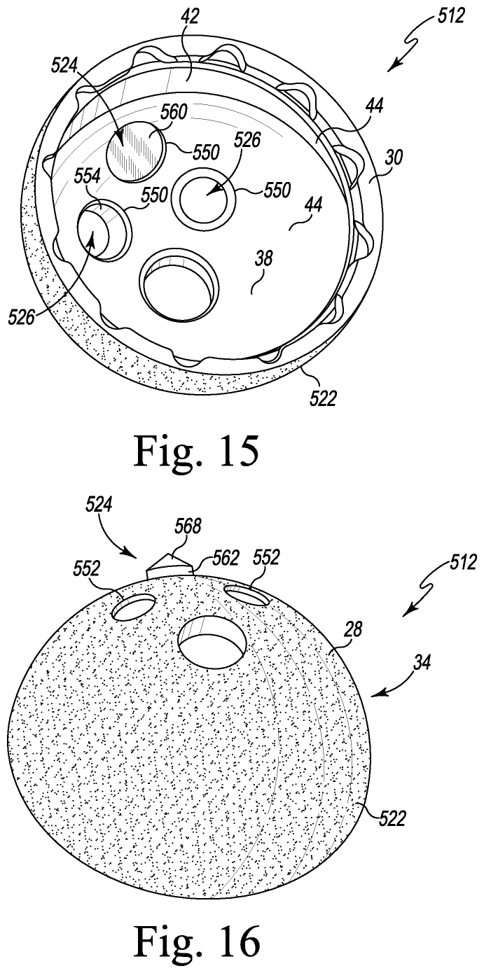

Referring now to FIGS. 15-16, another embodiment of an acetabular shell assembly (hereinafter shell assembly 512 is shown). A number of the features of the shell assembly 512 are the same as features shown and described above in regard to shell assembly 12. Those features are identified in FIGS. 15-16 using the same reference numbers. The shell assembly 512 includes a shell component 522 that is configured to receive the insert 14. As described in greater detail below, the shell assembly 512 also includes a plug 524, which is sized and shaped to be positioned in a mounting hole 526 defined in the shell component 522.

The shell component 522 has a distal rim 30 and an outer wall 32 that extends from the distal rim 30. The outer wall 32 includes a convex curved outer surface 34 and an annular outer surface 36 that extends from the distal rim 30 to the curved outer surface 34. In the illustrative embodiment, the convex curved outer surface 34 is semi-spherical, and the Porocoat® outer coating 28 covers the outer surface 34.

An inner wall 38 extends inwardly from the distal rim 30 to define a cavity 40 sized to receive the insert component 14. The inner wall 38 includes an annular inner surface 42 that is positioned opposite the annular outer surface 36 and a concave curved inner surface 44 that is connected to the annular inner surface 42.

Each mounting hole 526 has an inner opening 550 defined in the inner surface 44 and an outer openings 552 defined in the outer surface 34 and the outer coating 28. An inner wall 554 extends between the openings 550, 552 to define each mounting hole 526. In the illustrative embodiment, each mounting hole 526 is circular in shape. It should be appreciated that in other embodiments one or more of the mounting holes 526 may take other geometric forms such as, for example, an elongated, oblong slot. It should also be appreciated that in other embodiments the shell component may include only a single mounting hole.

The plug 524 illustratively includes a base 560 that is shaped to engage the inner wall 38 of each mounting hole 526. As shown in FIG. 16, the plug 524 includes a shaft 562 that extends outwardly from the base 560 to a tip 568 configured to engage the patient's acetabulum to provide additional fixation. In the illustrative embodiment, the tip 568 includes a point positioned beyond the outer surface 34 (including the coating 28). The plug 524 is formed from a polymeric material such as, for example, polyethylene. It should be appreciated that in other embodiments the plug 524 may be formed from an implant-grade metallic material.

To cover or seal the other mounting holes 526, it should be appreciated that plug similar to those described in the other embodiments above may be used. Additionally, the concepts associated with each of the plugs described herein may be utilized with shell components of varying configurations. For example, a plug with frangible seams similar to that described above in regard to FIG. 14 may be utilized to seal an oblong or otherwise elongated slot similar to that shown in FIG. 2.

While the disclosure has been illustrated and described in detail in the drawings and foregoing description, such an illustration and description is to be considered as exemplary and not restrictive in character, it being understood that only illustrative embodiments have been shown and described and that all changes and modifications that come within the spirit of the disclosure are desired to be protected.

There are a plurality of advantages of the present disclosure arising from the various features of the method, apparatus, and system described herein. It will be noted that alternative embodiments of the method, apparatus, and system of the present disclosure may not include all of the features described yet still benefit from at least some of the advantages of such features. Those of ordinary skill in the art may readily devise their own implementations of the method, apparatus, and system that incorporate one or more of the features of the present invention and fall within the spirit and scope of the present disclosure as defined by the appended claims.

1. An acetabular orthopaedic prosthesis, comprising:
an insert component including a cavity sized to receive a head of a femoral prosthetic component,
a shell including a convex outer surface shaped to engage a surgically-prepared surface of a patient's acetabulum, a concave inner surface that defines an aperture sized to receive the insert, and a plurality of mounting holes extending through the inner surface and the outer surface,
a plurality of plugs configured to be coupled to the shell, each plug being sized to be positioned in at least one of the mounting holes of the shell, and
a bone screw configured to be inserted through one of the plurality of plugs into the surgically-prepared surface to secure the shell to the patient's acetabulum.

2. The acetabular orthopaedic prosthesis of claim 1, wherein:
the plurality of mounting holes include a number of elongated slots,
the plurality of plugs include an elongated plug sized to be positioned in a first elongated slot of the number of elongated slots, the elongated plug including a guide hole sized to receive the bone screw, and
the guide hole has a diameter that is smaller than a length of the first elongated slot.

3. The acetabular orthopaedic prosthesis of claim 2, wherein:
the elongated plug is a first elongated plug having a first guide hole configuration that includes the guide hole sized to receive the bone screw, and
the plurality of plugs include a second elongated plug sized to be positioned in a second elongated slot of the number of elongated slots, the second elongated plug having a second guide hole configuration that is different from the first guide hole configuration.

4. The acetabular orthopaedic prosthesis of claim 3, wherein the second elongated plug includes a plurality of guide holes in the second guide hole configuration, each guide hole of the second elongated plug being sized to receive a bone screw.

5. The acetabular orthopaedic prosthesis of claim 3, wherein the second elongated plug is devoid of guide holes in the second guide hole configuration.

6. The acetabular orthopaedic prosthesis of claim 2, wherein the elongated plug includes a concave curved surface shaped to match a curvature of the concave inner surface of the shell.

7. The acetabular orthopaedic prosthesis of claim 1, wherein the shell is a metallic shell and each plug of the plurality of plugs is a polymeric plug.

8. The acetabular orthopaedic prosthesis of claim 1, wherein an the plurality of plugs include an insert plate sized to cover a number of mounting holes of the plurality of mounting holes, the insert plate including a concave curved surface shaped to match a curvature of the concave inner surface of the shell.

9. The acetabular orthopaedic prosthesis of claim 1, wherein:
the insert plate includes a number of openings sized to receive the bone screw and a peg removably positioned in and filling each opening, and
each opening of the insert plate is aligned with one of the mounting holes of the plurality of mounting holes.

10. The acetabular orthopaedic prosthesis of claim 1, wherein the plurality of plugs includes a first plug having a base sized to be positioned in one of the mounting holes of the shell and a shaft coupled to the base, the shaft being configured to extend outwardly from the outer surface of the shell to a tip configured to engage the patient's acetabulum.

11. The acetabular orthopaedic prosthesis of claim 1, wherein the plurality of plugs includes a first plug including a frangible seam configured to open to permit the bone screw to be inserted through the first plug into the surgically-prepared surface.

12. A method of implanting an acetabular orthopaedic prosthesis, the method comprising:
inserting a shell of the acetabular orthopaedic prosthesis into an acetabulum of a patient's pelvic bone, the shell including a mounting hole extending through an outer surface and an inner surface of the shell, and
inserting a bone screw through a plug positioned in the mounting hole into a surgically-prepared surface of the patient's acetabulum to secure the shell to the patient's acetabulum.

13. The method of claim 12, further comprising:
identifying a location on the surgically-prepared surface of the patient's acetabulum to insert the bone screw,
selecting a plug having a guide hole corresponding to the identified location, and
attaching the plug to the shell, wherein inserting the bone screw through the plug includes inserting the bone screw through the guide hole in the plug into the identified location on the surgically-prepared surface.

14. The method of claim 13, wherein the mounting hole is an elongated slot, and the guide hole has a diameter that is smaller than a length of the first elongated slot.

15. The method of claim 12, wherein:
the shell includes a plurality of mounting holes, and the plug includes a plate that covers a number of mounting holes of the plurality of mounting holes, and
inserting the bone screw through the plug includes selecting one mounting hole of the number of mounting holes and inserting the bone screw through the selected mounting hole into the surgically-prepared surface of the patient's acetabulum.

16. The method of claim 12, further comprising detaching a portion of the plug to expose a guide hole of the plug, wherein inserting the bone screw through the plug includes inserting the bone screw through the guide hole in the plug into the identified location on the surgically-prepared surface.

17. The method of claim 12, wherein inserting the shell of the acetabular orthopaedic prosthesis into the acetabulum of the patient's pelvic bone includes engaging a tip of the plug extending away from the outer surface of the shell with the surgically-prepared surface of the patient's acetabulum.

18. The method of claim 12, wherein inserting the bone screw through the plug includes engaging a tip of the bone screw with a frangible seam of the plug to open the seam and advancing the tip of the bone screw through the open seam.

19. An acetabular orthopaedic prosthesis, comprising:
an insert component including a cavity sized to receive a head of a femoral prosthetic component,
a shell including a convex outer surface shaped to engage a surgically-prepared surface of a patient's acetabulum, a concave inner surface that defines an aperture sized to receive the insert, and a mounting hole extending through the inner surface and the outer surface,
a plug configured to be coupled to the shell, the plug being sized to be positioned in the mounting hole of the shell and including a concave surface having a curvature that matches a curvature of the concave inner surface of the shell, and
a bone screw configured to be inserted through the plugs into the surgically-prepared surface to secure the shell to the patient's acetabulum.

20. The acetabular orthopaedic prosthesis of claim 19, wherein the plug includes a plurality of guide holes, each guide hole being sized to receive the bone screw.
